(19) United States
(12) Patent Application Publication
(10) Pub. No.: US 2008/0200902 A1
(43) Pub. Date: Aug. 21, 2008

(54) Connecting Device for Infusion Line

(76) Inventor: Hitoshi Mabuchi, Fukuroi-shi (JP)

Correspondence Address: TYCO HEALTHCARE - EDWARD S. JARMOLOWICZ, 15 HAMPSHIRE STREET, MANSFIELD, MA, 02048, US

(21) Appl. No.: 11/813,977

(22) PCT Filed: Jan. 12, 2006
(86) PCT No.: PCT/EP2006/000237

§ 371 Date: Apr. 22, 2008

(30) Foreign Application Priority Data
Jan. 14, 2005 (JP) 2005-007425

(51) Int. Cl. A61M 39/10

(52) U.S. Cl. 604/537

(57) ABSTRACT

The object is to keep a portion in the vicinity of a liquid discharge port in a clean state even at the time of priming or connection and to enable easy connection and disconnection of an infusion line without causing leakage of liquid from the liquid discharge port even when connection is released. This is solved by a tube (2) that forms a flow channel of a male connector (1) and is provided at its distal end portion (4) with a gas discharge portion (7) that blocks discharge of liquid and allows discharge of gas from the flow channel, a liquid discharge port (8) that allows discharge of liquid, and at a periphery of its distal portion (4) with shutter means (20) which opens the liquid discharge port (8) by a pressing force received from a male connector (30) when connected and restored to close the liquid discharge port (8) when the pressing force is released.

### TECHNICAL FIELD

The present invention relates to a connecting device for an infusion line provided in an infusion line or the like for allowing flow of blood or drug solution therethrough. In this specification, the term “rear end side” (or “base portion side”) represents the upstream side of the liquid flow when the drug solution or the like is administered via the connecting device in the invention, and the term “distal side” (or “distal portion side”) represents the downstream side.

### BACKGROUND ART

In the infusion line of a medical instrument, a closed system having an infection preventing function or an injection accident preventing function at a connecting portion is employed. For example, there is the one in which connection to the infusion line is achieved by providing a rubber-like resilient member, a so-called “septum”, for preventing infection, for allowing passage of a male connector tube therethrough at an infusion hole of a female connector of a three-way cock, and inserting the male connector tube through the septum on the female connector.

The state of the art includes [Patent Document 1] JP-T-8-500983 (pp.2, FIG. 2)

FIG. 11 below is a copy of FIG. 2 of JP-T-8-500983, and is a vertical cross-sectional view showing an example of a connecting device for an infusion line, which is composed of a male connector 70 and a female connector 80. The male connector 70 includes a male Luer connector 71 having a liquid discharge port 73 at the distal end, and the female connector 80 includes a septum 82 within a hollow retainer 81 so that the male Luer 71 can be fitted therein. The retainer 81 is formed with a projection 83 projecting outward from the outer peripheral surface, and the projection 83 is adapted to be engaged with a female screw in a tightening cylinder 72 of the male connector 70, whereby the male connector 70 and a female connector 80 can be engaged with and fixed to each other.

### DISCLOSURE OF THE INVENTION

### Problems to be Solved by the Invention

However, in the prior art connecting device for the infusion line having the liquid discharge port 73 at the distal end of the male Luer 71 of the male connector 70, liquid can leak from the liquid discharge port 73 after priming (air discharge) or may drip after the connector is disconnected and so easily become unclean, and protection of the portion near the liquid discharge port 73 is insufficient. A side surface of the male Luer 71 is exposed to the outside, whereby dust or fungus or leaked liquid may attach to the side surface and a peripheral portion thereof, and thus easily become unclean.

A technical object of the present invention is to keep the portion in the vicinity of a liquid discharge port in a clean state even when priming and connecting, and to achieve easy connecting and disconnecting to an infusion line without loss of liquid from the liquid discharge port even when the connection is released.

### Means for Solving the Problems

A connecting device for an infusion line according to the present invention is configured as follows. In a connecting device for an infusion line including a male connector and a female connector, the male connector and the female connector being provided at a connecting portion for an infusion line and forming a communication channel, a tube forming a flow channel of the male connector includes at the distal end portion a gas discharge portion for blocking discharge of liquid and allowing discharge of air from the flow channel and a liquid discharge port for allowing discharge of liquid therefrom, the tube is provided with shutter means around the distal end portion thereof, and the shutter means opens the liquid discharge port upon receiving a pressing force from the female connector when the tube is connected, and the shutter means returns to its former state and closes the liquid discharge port when the pressing force is released.

The gas discharge portion of the male connector includes an opening formed at the distal end of the male connector tube and a filter integrated with the tube and covering the opening so as to block discharge of liquid from the flow channel and allow discharge of gas therefrom, and the liquid discharge port is composed of a hole formed on a side wall of the distal portion of the male connector tube.

The gas discharge portion of the male connector includes a first side hole formed on the side wall of the distal end portion of the male connector tube, and a filter integrated with the tube and covering the first side hole so as to block discharge of liquid from the flow channel and allow discharge of gas therefrom, and the liquid discharge port is composed of a second side hole formed upstream of the first side hole on the side wall at the distal end portion of the male connector tube.

Shutter dislodging preventing means for preventing dislodging of the shutter means from the male connector tube is provided.

The male connector tube is provided with a protruding portion the shape of an outwardly extending flange, protruding at the axial midsection of the male connector tube, and an outer cylinder extending axially from the outer peripheral edge of the protruding portion toward the distal end and surrounding the distal end portion of the tube. The shutter dislodging preventing means includes a groove-like recess formed on the inner surface of the outer cylinder so as to extend axially, and a stopper projecting from the shutter means, capable of being slidably fitted in the groove-like recess and engaging with the distal end surface of the groove-like recess.

Holding means for holding the connection between the male connector and the female connector is provided.

The rear end portion of the female connector is formed so as to be capable of being fitted into the outer cylinder of the male connector, and the holding means includes a hook-shaped notch formed by forming a groove at the front end surface of the outer cylinder of the male connector and a projection formed on an outer surface of the rear end portion of the female connector so as to project therefrom and be capable of engaging with the hook-shaped notch of the male connector.

An infusion hole of the female connector that allows insertion of the male connector tube is provided with a rubber-like resilient member having a slit configured to allow insertion of the male connector tube and have a thickness allowing penetration of the liquid discharge port of the male connector tube.

The male connector is integrally provided at the distal end of a syringe.

### Advantages of the Invention

According to the present invention, it is possible for only gas to be discharged from the inside of the tube forming the flow channel of the male connector while blocking discharge of liquid therefrom (during priming), and hence the interior of the tube can be filled with liquid. Therefore, even though the male connector is disconnected, little leakage of liquid occurs. Therefore, attachment of fungus or dust occurs mainly on the outer surface of the shutter means, and can be removed easily by wiping. Consequently, the liquid discharge port or the peripheral portion thereof can be kept constantly in a clean state.

### BRIEF DESCRIPTION OF THE DRAWINGS

FIG. 1 shows a left side view, a front view, and a right side view of a male connector of a connecting device for an infusion line according to a first embodiment of the present invention.

FIG. 2 is a vertical cross-sectional view of the male connector of the connecting device for an infusion line according to the first embodiment of the present invention.

FIG. 3 is a left side view, a front view, and a right side view of a female connector of the connecting device for an infusion line according to the first embodiment of the present invention.

FIG. 4 is a vertical cross-sectional view of the female connector of the connecting device for an infusion line according to the first embodiment of the present invention.

FIG. 5 is a vertical cross-sectional view of the male connector and the female connector in a connected state of the connecting device for an infusion line according to the first embodiment of the present invention.

FIG. 6 is a vertical cross-sectional view of a male connector of a connecting device for an infusion line according to a second embodiment of the present invention.

FIG. 7 is a vertical cross-sectional view showing a connected state between the male connector and a female connector of the connecting device for an infusion line according to the second embodiment of the present invention.

FIG. 8 shows a left side view, a front view, and a right side view of a syringe provided with a male connector of a connecting device for an infusion line according to a modification of the present invention.

FIG. 9 is a vertical cross-sectional view of the syringe of the connecting device for an infusion line according to the modification of the present invention.

FIG. 10 is a vertical cross-sectional view showing a state before connection and after connection of an infusion portion which is a principal portion of a connecting device for an infusion line according to another modification of the present invention.

FIG. 11 is a vertical cross-sectional view showing a connecting device for an infusion line in the related art.

### REFERENCE NUMERALS

1 male connector

2 infusion tube (tube)

4 infusion portion (distal end portion of tube)

7, 7A gas discharge portion

8 liquid discharge port (hole)

8A liquid discharge port (second side hole)

9 opening of gas discharge portion

11, 11A filter of gas discharge portion

12 protruding portion

13 outer cylinder

15 shutter dislodging preventing means

16 groove-like recess

17 distal end surface of groove-like recess

18 stopper

20, 20A shutter means

29 hook-shaped notch

30 female connector

33 single cylindrical portion (rear end portion of female connector)

34 projection

35 holding means

37 interior space (infusion hole of female connector)

38 slit

39 septum (rubber-like resilient member)

50 syringe

60 first side hole (opening of gas discharge portion)

### BEST MODE FOR CARRYING OUT THE INVENTION

### First Embodiment

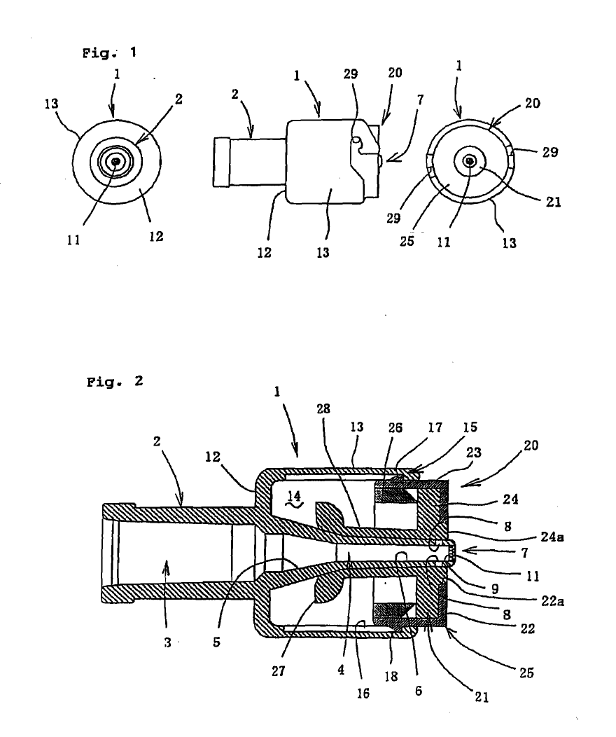

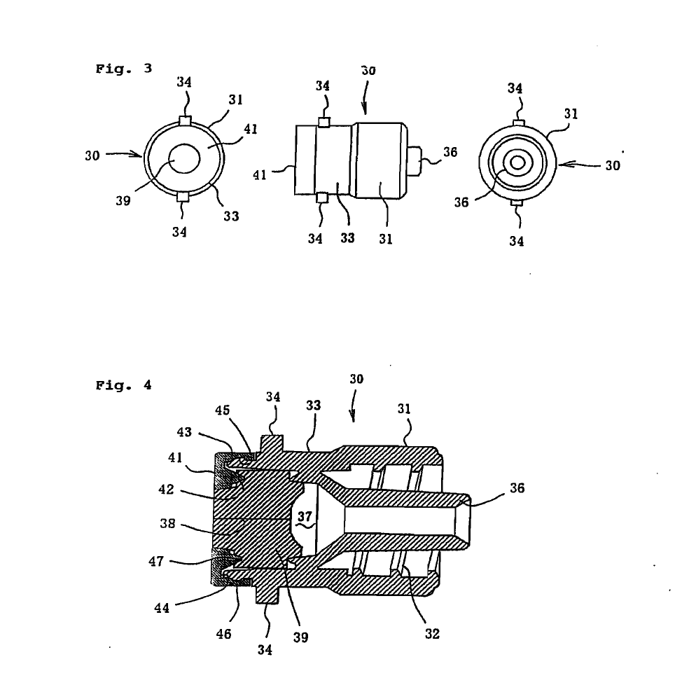

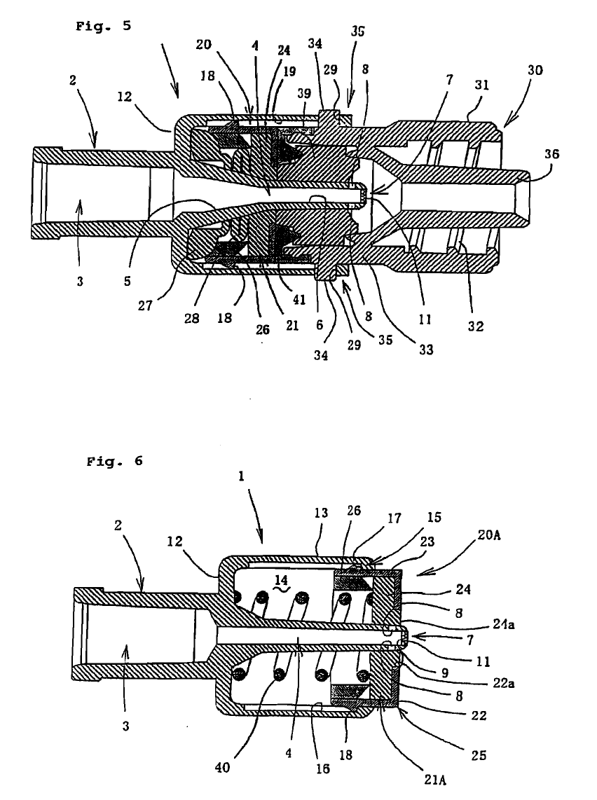

FIG. 1 to FIG. 5 show a connecting device for an infusion line according to a first embodiment of the present invention. FIG. 1 shows a left side view, a front view, and a right side view of a male connector; FIG. 2 is a vertical cross-sectional view of the male connector; FIG. 3 is a left side view; a front view, and a right side view of a female connector; FIG. 4 is a vertical cross-sectional view of the female connector; and FIG. 5 is a vertical cross-sectional view of the male connector and the female connector in a connected state.

The connecting device for an infusion line according to this embodiment includes a male connector 1 and a female connector 30 formed of synthetic resin that are provided at a connecting portion to the infusion line and define a communication channel. The male connector 1 is provided with a tube defining a flow channel to which liquid such as drug solution is infused, that is, an infusion tube 2 as shown in FIG. 2. The infusion tube 2 includes a cylindrical introduction portion 3 positioned on its base side, for connecting a tube or a syringe, and an infusion portion 4 positioned on the distal portion side and having a diameter smaller than the introduction portion 3. The infusion portion 4 further includes a conical tapered infusion portion 5 positioned by the introduction portion 3, gradually reducing in diameter toward the distal end, and a substantially cylindrical distal infusion portion 6 positioned on the distal side and gradually reducing in diameter toward the distal end.

The infusion tube 2 is provided at its distal end portion with a gas discharge portion 7 that blocks discharge of liquid from the flow channel and allows discharge of gas therefrom, and a liquid discharge port 8 that allows discharge of liquid, and around its distal end with shutter means 20 which opens the liquid discharge port 8 when it receives a pressing force from the female connector 30 at the time of connection and blocks the liquid discharge port 8 when it returns to its original state upon release of the pressing force.

More specifically, the gas discharge portion 7 includes an opening 9 formed at the distal end of the infusion tube 2, and a filter 11 integrated with the infusion tube 2 by fusion or adhesion and covering the opening 9 so as to block discharge of liquid from the flow channel and allow discharge of gas therefrom. Here, a moisture permeable/water-proof film formed mainly of polypropylene or polyethylene and having innumerable micron-sized fine holes formed thereon is used for the filter 11. The moisture permeable/water-proof film has the characteristic that it permits the passage of gas such as air but blocks liquid such as water. The liquid discharge port 8 is composed of a plurality of holes formed on a side wall of the distal end portion of the infusion tube 2 of the male connector 1.

The infusion tube 2 is formed with an protruding portion 12 which protrudes in the shape of an outward flange in the axial midsection of infusion tube 2, and an outer cylinder 13 that extends from the outer peripheral edge of the protruding portion 12 axially toward the distal end so as to surround the distal end portion of the infusion tube 2, that is, the infusion portion 4. Provided between the outer cylinder 13 and the infusion portion 4 of the infusion tube 2 is a space 14, and the shutter means 20 is mounted in the space 14 via shutter dislodging preventing means 15 so as to be capable of axial sliding movement. The shutter dislodging preventing means 15 is means that prevents dislodging of the shutter means 20 from the infusion portion 4, and is composed of a groove-like recess 16 formed on the inner surface of the outer cylinder 13 extending in the axial direction, and a stopper 18 that is formed projecting from the shutter means 20 and is capable being slidably fitted in the groove-like recess 16 and engaging with the distal end surface 17 of the groove-like recess 16. The stopper 18 also has a function to guide the entire shutter means 20 in the space 14 of the outer cylinder 13 in the axial direction by being slid in the groove-like recess 16.

The shutter means 20 includes, as shown in FIG. 2, a cylindrical shutter body 21 formed as a resilient member for covering the infusion tube 2 from the distal peripheral side wall of the distal infusion portion 6 to the distal, reduced diameter portion of the tapered infusion portion 5, a sliding member 25 having an inwardly extending flange-shaped base 22 at its distal end and a cylindrical portion 23 extending from the outer peripheral edge of the base 22 continuously toward the rear in the axial direction so as to accommodate a valve forming portion 24 at the distal end of the shutter body 21, and a fixed member 26 arranged at the rear of the cylindrical portion 23 of the sliding member 25, that is, to the rear of the valve forming portion 24 of the shutter body 21 accommodated in the cylindrical portion 23, for pushing and fixing the valve forming portion 24 to the distal end (against the base 22 of the sliding member 25), wherein the stopper 18 of the shutter dislodging preventing means 15 is formed projecting from the outer surface of the cylindrical portion 23 of the sliding member 25. An annular projection 24a formed on the valve forming portion 24 of the shutter body 21 so as to project therefrom is fitted into a hole 22a in the base 22 of the sliding member 25, and the distal end surface of the annular projection 24a and the distal end surface of the base 22 of the sliding member 25 are formed to be flush with each other so as to come into abutment with a rear end surface of the female connector 30.

The rear end portion of the shutter body 21 protrudes radially outward so as to serve as a tightening portion 27, and the inner peripheral surface of the tightening portion 27 is formed into a tapered shape extending along the outer peripheral surface of the distal, reduced diameter portion of the tapered infusion portion 5. The portion of the shutter body 21 near the axial center is formed into a cylindrical portion 28 which can be deformed into an accordion or concertina as shown in FIG. 5 when compressed, and the distal portion of the shutter body 21 is expanded in a cylindrical shape and formed as the valve forming portion 24. Thus, when the shutter body 21 receives a pressing force exceeding a predetermined level toward the base side of the infusion portion 4 and is forced to be moved, the liquid discharge port 8 of the distal infusion portion 6 is freed from occlusion by the shutter body 21, that is, from the valve forming portion 24. When from this opened state the pressing force is released, the shutter body 21 is restored to the distal side by the restoration force, and the liquid discharge port 8 of the distal infusion portion 6 is covered (occluded) by the valve forming portion 24 and blocked thereby. The predetermined pressing force here is determined by the friction between the shutter body 21 and the infusion portion 4 and the taper angle of the tapered infusion portion 5.

The male connector 1 is formed with hook-shaped notches 29 formed on the front end surface on the outer cylinder 13 at a plurality of positions along the circumference.

The female connector 30 is formed as double tubular portion at the distal side of the main body, and a female screw 32 is provided on the inner peripheral surface of an outer cylinder 31 of the double cylindrical portion, so that connection to a port or the like of a three-way cock is achieved by screwing in this female screw 32. The rear end side of the main body of the female connector 30 is formed as a single cylindrical portion 33 continuing from the outer cylinder 31, and the single cylindrical portion 33 is provided with projections 34 that can engage with the hook-shaped notches 29 (FIG. 1) on the side of the male connector 1 at a plurality of positions on an outer peripheral surface thereof along the circumference. Accordingly, the hook-shaped notches 29 on the side of the male connector 1 and the respective projections 34 on the side of the female connector 30 constitute holding means 35 for maintaining a connection between the male connector 1 and the female connector 30. An infusion tube 36 that corresponds to the inner cylinder of the double cylindrical portion on the distal side has a substantially Y-shaped cross section that opens out from the distal end toward the rear end, and the rear end portion is integrated with the single cylindrical portion 33. A septum 39 formed of rubber-like resilient member with slits 38 that allow insertion of the distal infusion portion 6 of the male connector 1 and set to a thickness that allows penetration of the liquid discharge port 8 of the distal infusion portion 6 when connecting the male connector 1, and configured so that the septum 39 and a holding member 41 abut with the shutter means 20 when connected, is filled into the interior space 37 of the single cylindrical portion 33 that corresponds to a rear end opening, and is prevented from dislodging from the interior space 37 by the holding member 41.

As shown in FIG. 4, the holding member 41 is formed into the shape of two rings whose rear edges are connected to each other, the distal side of the inner ring 42 formed with a taper on the inner and outer peripheral surfaces so as to form an acute angle in cross section. Formed between the inner ring 42 and the outer ring 43 is an annular groove 44, so that the holding member 41 is mounted to the main body by fitting the annular groove 44 to the rear end of the main body of the female connector 30. The inner peripheral surface of the outer ring 43 of the holding member 41 is provided with an annular recess 45, and the outer peripheral surface at the rear end portion of the main body facing the annular recess 45 when the holding member 41 is mounted is provided with an annular projection 46 that can engage with the annular recess 45 when the holding member 41 is mounted, whereby the holding member 41 can be stably mounted on the female connector body.

The septum 39 is formed with an annular groove 47 on the peripheral portion of its rear end surface, in which can be fitted the inner ring 42 of the holding member 41. The holding member 41 prevents the septum 39 from dislodging from the female connector body.

In order to connect an infusion line via the male connector 1 and the female connector 30 of the connecting device according to this embodiment configured as described above, infusion solution is filled into the infusion portion 4 from the introduction portion 3 in the infusion tube 2 of the male connector 1 as a first step. Accordingly, only air is discharged through the filter 11 at the distal end of the infusion tube 2 and priming is completed.

Subsequently, the distal surface of the annular projection 24a and the distal surface of the bottom 22 of the shutter means 20 of the male connector 1 are brought into abutment and pressed against the rear end surface of the female connector 30 positioned, for example, on the side of the three-way cock. Accordingly, the shutter means 20 is guided in the groove recess 16 on the outer cylinder 13 and retracts in the space 14. At this time, the distal infusion portion 6 of the infusion tube 2 is inserted into the slits 38 from the rear side of the septum 39 of the female connector 30, moving relative to the shutter means 20. When the shutter means 20 is further retracted, the tightening portion 27 of the shutter body 21 comes into abutment with the protruding portion 12, which corresponds to the bottom of the outer cylinder 13. When the shutter means 20 is further pushed in this state, the cylindrical portion 28 of the shutter body 21 is deformed into an accordion shape. Then, from this state, by rotating the male connector 1 while pushing the same, the respective projections 34 of the female connector 30 are fitted and engaged to the respective hook-shaped notches 29 of the male connector 1 as shown in FIG. 5, and the liquid discharge port 8 of the distal infusion portion 6 of the male connector 1 is fixed beyond the septum 39 of the female connector 30, introduced into the interior space 37.

In this manner, since the shutter means 20 of the male connector 1 comes into abutment with the rear end surface of the female connector 30 and is pushed and shortened, the male connector 1 and the female connector 30 are connected with communication between the two, the liquid discharge port 8 of the distal infusion portion 6 of the male connector 1 opening only in the interior space 37 in the female connector 30 and not opening to the outside air. In the case in which the connected male connector 1 and female connector 30 become disconnected by mistake, the shutter means 20 is returned to the distal side by the restoration force of the shutter body 21 when the male connector 1 separates, so that the liquid discharge port 8 of the distal infusion port 6 of the male connector 1 is closed by the valve forming portion 24 of the shutter body 21, whereby discharge of liquid is restrained.

In other words, when the male connector 1 and the female connector 30 are connected with communication between the two, the shutter means 20 is in the state of retraction, and the tightening portion 27 of the shutter body 21 is forcedly opened by the tapered surface of the tapered infusion portion 5 which increases in diameter toward the base. Furthermore, since the cylindrical portion 28 of the shutter body 21 is deformed into an accordion shape and hence is compressed in the axial direction, restoration force in the radially inward direction on the tapered surface is generated, and a component force thereof acts to push the shutter means 20 toward the distal end. Therefore, when the male connector 1 is pulled out from the female connector 30, the shutter body 21 slides along the tapered surface toward the small diameter side of the tapered infusion portion 5, and accordingly, the shutter means 20 is guided by the groove-like recess 16 of the outer cylinder 6 to move toward the distal end, and thus the valve forming portion 24 of the shutter body 21 closes the liquid discharge port 8 of the distal infusion portion 6, whereby the state shown in FIG. 1 is restored.

In this manner, according to this embodiment, it is possible merely by filling infusion solution into the infusion portion 4 from the introduction portion 3 in the infusion tube 2 of the male connector 1 to discharge air through the filter 11 at the distal end of the infusion tube 2, and priming can be completed. Also, since the tapered infusion portion 5 is provided at the infusion portion 4 of the male connector 1 so as to provide a resilient force in the radial direction (a component of which acts toward the distal end) to the tightening portion 27 of the shutter body 21, and the cylindrical portion 28 which is deformable in the accordion shape is provided in the shutter body 21 to provide an axial resilient force, an urging force on the shutter means 20 toward the distal end can be generated, so that the liquid discharge port 8 of the distal infusion portion 6 of the male connector 1 can normally be closed by the shutter means 20. Therefore, leakage of liquid at the time of priming can be minimized, whereby diffusion of liquid which tends to be a breeding ground of infection can be prevented. In addition, ingress of dust or the like in the interior space 14 of the outer cylinder 13 can be prevented, so that the infusion portion 4 and the periphery thereof can be maintained constantly in a clean state.

The closed state of the liquid discharge port 8 in the normal state can be maintained by the shutter dislodging preventing means 15 and the shutter means 20 is prevented from dislodging from the outer cylinder 13.

When necessary, it is possible merely by applying a force in the direction toward the base side to the shutter means 20 to open the liquid discharge port 8 of the distal infusion portion 6, and hence usability is improved.

Since the connected state between the male connector 1 and the female connector 30 can be maintained by the holding means 35, reliability can be secured easily.

### Second Embodiment

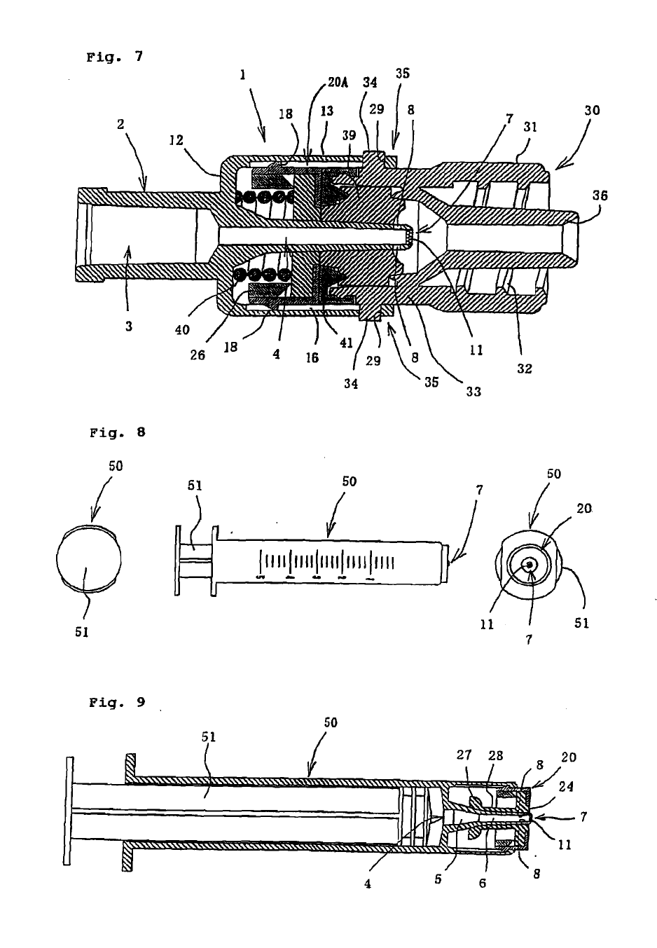

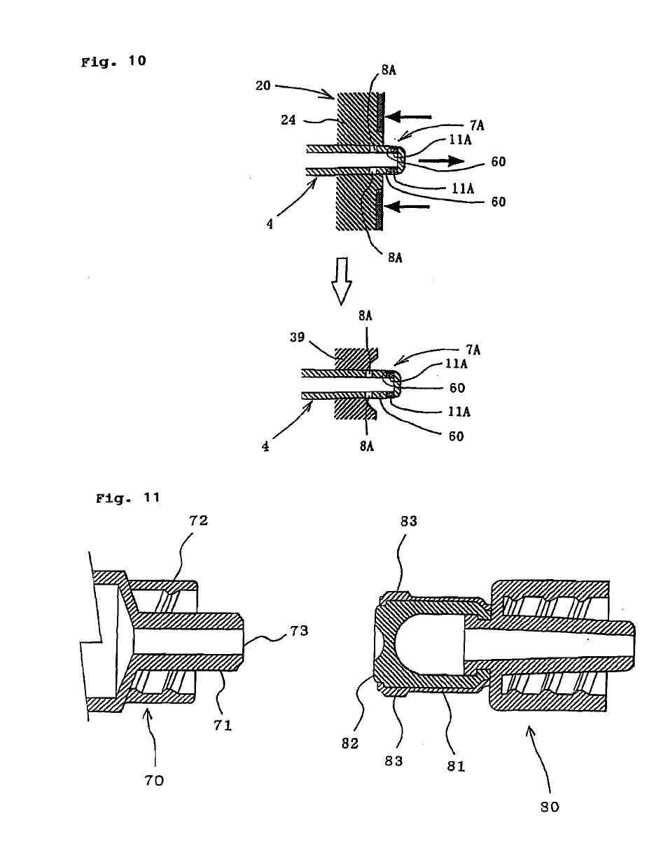

FIG. 6 and FIG. 7 both show a connecting device for an infusion line according to second embodiment of the present invention. FIG. 6 is a vertical cross-sectional view of a male connector, and FIG. 7 is a vertical cross-sectional view showing a connected state between the male connector and a female connector. In these drawings, the same or corresponding parts as/to those in the first embodiment are represented by the same reference numerals.

The connecting device for an infusion line in the second embodiment is different from the above-described first embodiment in the method of generating a restoration force in the shutter means 20A provided on the distal end portion of the infusion tube 2, opening the liquid discharge port 8 with pressing force received from the female connector 30 when connected, and returning to the original state when the pressing force is released, thereby closing the liquid discharge port 8.

Specifically, in the first embodiment (FIG. 2) as described above, the resilient force (restoration force) of the shutter body 21 is used for restoring the shutter means 20 to its original position, that is, the position where the stopper 18 of the shutter dislodging preventing means 15 engages the distal end surface 17 of the groove-like recess 16. However, in the second embodiment, the shutter means 20A is restored to the original position using a spring force.

More specifically, a shutter body 21A, formed of a resilient member, of the shutter means 20A is composed only of the column-shaped valve forming portion 24, and a coil spring 40 for urging the valve forming portion 24 constantly in the distal end direction so that the stopper 18 of the shutter dislodging preventing means 15 engages with the distal end surface 17 of the groove-like recess 16 is arranged between the back surface of the valve forming portion 24 and the inner surface of the protruding portion 12 which corresponds to the bottom of the outer cylinder 13. Other structures including the gas discharge portion 7 and the holding means 35 are the same as those in the first embodiment described above.

In the connecting device for an infusion line in this embodiment, in addition to the same effects as in the first embodiment, the resilient force (restoration force) can easily be changed merely by changing the spring constant. Therefore, there are the advantages that design freedom including selection of material of the shutter body 21 is increased, and operation is stable.

FIG. 8 and FIG. 9 both show a connecting device for an infusion line according to a modification of the present invention. FIG. 8 shows a left side view, a front view, and a right side view of a syringe provided with a male connector, and FIG. 9 is a vertical cross-sectional view of the same syringe. In these drawings, the same parts as those in the first embodiment described above are represented by the same reference numerals.

In the connecting device for an infusion line in this modification, basically, the introduction portion 3 of the infusion tube 2 of the male connector 1 described in conjunction with the first embodiment (FIG. 2) is eliminated and the outer cylinder 13 is extended rearward to constitute a syringe 50. This modification can be applied to the connecting device in the first embodiment and the second embodiment described above.

In the connecting device for an infusion line in this modification, by pressing a plunger 51 of the syringe 50 and filling infusion solution from the syringe 50 into the infusion portion 4 in the state in which the liquid discharge port 8 is closed and protected by the shutter means 20, only air is discharged through the filter 11 on the gas discharge portion 7 at the distal end of the infusion portion 4 and hence the priming is completed.

In this manner, in the connecting device for an infusion line according to this modification, since the priming can be performed with the liquid discharge port 8 closed, a protective cap or the like which was necessary for the syringe in the related art is not necessary any longer, and hence usability is improved.

FIG. 10 is a vertical cross-sectional view showing a state before connection and after connection of the infusion portion which is a principal portion of the connecting device for an infusion line according to another modification of the present invention. In the drawing the same parts as those in the first embodiment described above are represented by the same reference numerals.

According to the connecting device for an infusion line according to this modification, a gas discharge portion 7A of a male connector is composed of a plurality of first side holes 60 formed on a side wall of the distal portion of the infusion portion 4 of the male connector and a filter 11A integrated to the infusion portion 4 and covering the respective first side holes 60 by fusion or adhesion so as to block discharge of liquid from the flow channel and allow discharge of gas therethrough, and a liquid discharge port 8A is composed of second side holes formed upstream of the first side holes 60 on the side wall of the distal portion of the infusion portion 4 of the male connector. This modification may be applied to either the first or second embodiment, and the modifications (FIG. 8, FIG. 9) described above.

In the connecting device for an infusion line in this modification, the distal end of the infusion portion 4 projects from the shutter means 20 to an extent in which the respective gas discharge portions 7A are opened in the normal state and, in this state, the respective liquid discharge ports 8A are closed by the valve forming portion 24 of the shutter means 20. In other words, the priming can be performed with the respective liquid discharge ports 8A closed.

When connecting with the female connector 30, the septum 39 is penetrated to the portion where there are the respective liquid discharge ports 8A, so that the respective liquid discharge ports 8A are opened, allowing infusion.

In the connecting device for an infusion line in this modification, the distal end of the infusion portion 4 is completely closed and it looks like a solid shaft in appearance. Therefore, resistance applied when penetrating the septum 39 can be reduced, and hence the usability is improved.

1. A connecting device for an infusion line comprising:
a male connector; and
a female connector,
the male connector and the female connector being provided at a connecting portion for an infusion line and defining a communication channel,
and wherein:
a tube defining a flow channel of the male connector includes a gas discharge portion which resists discharge of liquid yet permits discharge of air from the flow channel and also includes a liquid discharge port which permits discharge of liquid therefrom at a distal end portion thereof,
the tube is provided with shutter means around the distal end portion thereof, and that is movable between a closure disposition in which it occludes the liquid discharge port and an open disposition in which it permits liquid discharge from the liquid discharge port
the shutter means is movable to the open disposition by a pressing force received from the female connector when connected and is restored to the original closure disposition when the pressing force is released.

2. The connecting device for an infusion line according to claim 1, wherein the gas discharge portion of the male connector comprises an opening formed at a distal end of the male connector tube and a filter integrated with the tube and covering the opening so as to block discharge of liquid from the flow channel and allow discharge of gas therefrom, and the liquid discharge port is composed of a hole formed on a side wall of the distal portion of the male connector tube.

3. The connecting device for an infusion line according to claim 1 or 2, wherein the gas discharge portion of the male connector comprises a first side hole formed on the side wall of the distal end portion of the male connector tube, and a filter integrated with the tube and covering the first side hole so as to block discharge of liquid from the flow channel and allow discharge of gas therefrom, and the liquid discharge port is composed of a second side hole formed upstream of the first side hole on the side wall at the distal end portion of the male connector tube

4. The connecting device for an infusion line according to any one of claims 1 to 3, comprising shutter dislodging preventing means for preventing dislodging of the shutter means from the male connector tube.

5. The connecting device for an infusion line according to claim 4,
wherein the male connector tube comprises a protruding portion protruding at an axial midsection of the male connector tube in the shape of radially outwardly extending flange, and an outer cylinder portion extending axially from the outer peripheral edge of the protruding portion toward the distal end for surrounding the distal end portion of the tube, and
the shutter dislodging preventing means comprises:
a groove-like recess formed on the inner surface of the outer cylinder so as to extend axially and
a stopper projecting from the shutter means capable of being slidably fitted in the groove-like recess and engaging with the distal end surface of the groove-like recess.

6. The connecting device for an infusion line according to any one of claims 1 to 5 comprising holding means for holding a connected state between the male connector and the female connector.

7. The connecting device for an infusion line according to claim 6,
wherein the rear end portion of the female connector is formed so as to be capable of being fitted into the outer cylinder of the male connector, and
the holding means comprises:
a hook-shaped notch formed by forming a groove from the front end of the outer cylinder of the male connector and
a projection formed on the outer surface of the rear end portion of the female connector so as to project therefrom and being capable of engaging with the hook-shaped notch of the male connector.

8. The connecting device for an infusion line according to any one of claims 1 to 7, wherein an infusion hole of the female connector that allows insertion of the male connector tube is provided with a rubber-like resilient member set to have a slit for allowing insertion of the male connector tube and thickness which allows penetration of the liquid discharge port of the male connector tube.

9. The connecting device for an infusion line according to any one of claims 1 to 8, wherein the male connector is integrally provided at a distal end of a syringe.
